(19) United States
(12) Patent Application Publication
(10) Pub. No.: US 2009/0170932 A1
(43) Pub. Date: Jul. 2, 2009

(54) DISINFECTANT COMPOSITIONS, METHODS AND SYSTEMS

(75) Inventors: Dinesh AGGARWAL, Franklin, MA (US); Chirag Shah, N. Attleboro, MA (US)

Correspondence Address: BUCHANAN INGERSOLL & ROONEY, PC, P.O. BOX 1404, ALEXANDRIA, VA, 22313-1404, US

(73) Assignee: Tyco Healthcare Group LP, Mansfield, MA (US)

(21) Appl. No.: 12/347,073

(22) Filed: Dec. 31, 2008

Related U.S. Application Data
(60) Provisional application No. 61/006,201, filed on Dec. 31, 2007.

(51) Int. Cl. A61K 31/375; A61L 2/18

(52) U.S. Cl. 514/474; 422/28

(57) ABSTRACT

Disinfectant compositions comprising N-Acetyl Cysteine (NAC) and Vitamin C are disclosed. The disinfectant compositions have also demonstrated activity in inhibiting as well as killing micro-organisms responsible for generating biofilms. They are safe for human and medical uses and may be used as prophylactic preparations to reduce the proliferation of and/or eliminate existing or established infections.

### BACKGROUND

Infections are a significant problem in many fields where sanitary conditions are important, such as in healthcare. Problematic infections may arise from bacterial, fungal, amoebic, protozoan and/or viral organisms. Challenges are encountered both in preventing infection, and in reducing or eliminating the infection once it is established. Infected environments may include surfaces of objects, fluids and fluid conduits and/or humans or animals.

Alcohol solutions and isopropyl alcohol wipes are commonly used to disinfect surfaces and have been shown to have antibacterial activity. The most effective inhibitory anti-microbial effect is seen with 70% isopropanol solutions. Alcohol solutions at this concentration are quite expensive and rapidly evaporate, which substantially diminishes their efficacy and increases their cost. Moreover, although isopropanol solutions may be used for surfaces, including human skin, and in a variety of medical applications, alcohol solutions of this concentration cannot be administered to humans, for medical purposes, other than topically.

In the healthcare field, infections of various types and causes are common and often result in longer hospital stays, producing higher hospital costs. Even worse, over 90,000 patient deaths annually are attributed to nosocomial infections—that is, infections acquired at a hospital or in another healthcare environment. Surveillance for nosocomial infection has become an integral part of hospital practice. Studies conducted more than 20 years ago by the Centers for Disease Control and Prevention (CDC) documented the efficacy of these surveillance activities in reducing nosocomial infection occurrence. Despite the attention paid to problems of nosocomial infection, however, infection rates have not been dramatically reduced, and nosocomial infections remain a substantial risk and a substantial health concern.

One problematic source of infections in the medical and veterinary fields is found in catheters, and particularly in in-dwelling catheters. Catheters have become essential in the management of critical care patients, yet the inside of a catheter is often the major source of infection. Catheters are used for delivery of fluids, blood products, drugs, nutrients, hemodialysis, hemofiltration, peritoneal dialysis, retrieval of blood samples, monitoring of patient conditions, etc. Transcutaneous catheters often become infected through skin penetration of the catheter. It has been found that seventy percent (70%) of all nosocomial bloodstream infections occur in patients with central venous catheters. Daouicher et al. 340, 1-8, NEW ENGLAND JOURNAL OF MEDICINE (1999).

In particular, during some procedures, a catheter must be implanted in, and remain implanted in, a patient for a relatively long period of time, e.g. over thirty days. Intravenous (IV) therapy catheters and urinary catheters typically remain implanted for a substantial period of time. As a result of trauma to the areas of insertion, and pain to the patients, such catheters can't be removed and implanted frequently. Catheter-borne bacteria are implicated as a primary source of urinary tract infections. Patients who receive a peripherally inserted central catheter during pregnancy have also been found to be at significant risk for infectious complications. “Complications Associated With Peripherally Inserted Central Catheter Use During Pregnancy” AM. J. OBSTET. GYCOL. 188(5):1223-5 May 2003. In addition, central venous catheter infection, resulting in catheter related sepsis, has been cited as the most frequent complication during home parenteral nutrition. CLINICAL NUTRITION, 21(1):33-38, 2002. Because of the risk of infections, catheterization may be limited to incidences when the procedure is absolutely necessary. This seriously compromises patient health.

After most prescribed access medical procedures involving a catheter, the catheter is flushed with saline and then filled with a liquid, such as saline or a heparin solution, to prevent blood from clotting inside of the catheter, to inhibit the patient's blood from backing up into the catheter, and to prevent gases from entering the catheter. The liquid that is used to flush the catheter is referred to as a “lock-flush,” and the liquid used to fill the catheter following flushing or during periods of non-use is referred to as a “lock” solution.

Traditionally, catheters have been locked with normal saline or heparin solutions. Heparin and saline are sometimes used in combination. Normal saline is generally used to lock short term peripheral intravenous catheters, but saline has no anticoagulant or antimicrobial activity. Heparin solutions are generally used to lock vascular catheters. Heparin has anticoagulant activity but it does not function as an antimicrobial and does not prevent or ameliorate infections. There are also strong indications that heparin in lock solutions may contribute to heparin-induced thrombocytopenia, a serious bleeding complication that occurs in a subset of patients receiving heparin injections.

Catheter locking solutions comprising taurolidine, citric acid and sodium citrate have been proposed. A recent publication (Kidney International, September 2002) describes the use of a 70% alcohol solution as a lock solution for a subcutaneous catheter port. The use of alcohol as a lock solution is questionable, since it is not an anticoagulant, and since there would be risks associated with this solution entering the bloodstream. There is also no evidence that the inventors are aware of that indicates that a 70% alcohol solution has any biofilm eradication activity.

An emerging trend and recommendation from the Center for Infectious Disease (CID) is to treat existing catheter infections systemically with either a specific or a broad range antibiotic. Use of an antibiotic in a lock solution to prevent infection is not recommended. The use of antibiotics to treat existing catheter infections has certain risks, including: (1) the risk of antibiotic-resistant strains developing; (2) the inability of the antibiotic to kill sessile, or deep-layer biofilm bacteria, which may require the use of antibiotics at toxic concentrations; and (3) the high cost of prolonged antibiotic therapy. Catheters coated with a disinfectant or antibiotic material are available. These coated catheters may only provide limited protection for a relatively short period of time.

In general, free-floating organisms may be vulnerable to antibiotics. However, bacteria and fungi may become impervious to antibiotics by attaching to surfaces and producing a slimy protective substance, often referred to as extra-cellular polymeric substance (EPS), polysaccharide covering or glycocalyx. As the microbes proliferate, more than 50 genetic up or down regulations may occur, resulting in the formation of a more antibiotic resistant microbial biofilm. One article attributes two-thirds of the bacterial infections that physicians encounter to biofilms. SCIENCE NEWS, 1-5 Jul. 14, 2001.

Biofilm formation is a genetically controlled process in the life cycle of bacteria that produces numerous changes in the cellular physiology of the organism, often including increased antibiotic resistance (of up to 100 to 1000 times), as compared to growth under planktonic (free floating) conditions. As the organisms grow, problems with overcrowding and diminishing nutrition trigger shedding of the organisms to seek new locations and resources. The newly shed organisms quickly revert back to their original free-floating phase and are once again vulnerable to antibiotics. However, the free-floating organism may enter the bloodstream of the patient, creating bloodstream infections, which produce clinical signs, e.g. fever, and more serious infection-related symptoms. Sessile rafts of biofilm may slough off and may attach to tissue surfaces, such heart valves, causing proliferation of biofilm and serious problems, such as endocarditis.

In industrial settings, the formation of biofilms is very common and is generally referred to as biofouling. For example, biofilm growth on mechanical structures, such as filtration devices, is a primary cause of biological contamination of drinking water distribution systems. Biofilm formation in industrial settings may lead to material degradation, product contamination, mechanical blockage and impedance of heat transfer in processing systems. Biofilm formation and the resultant contamination is also a common problem in food preparation and processing facilities.

To further complicate matters, conventional sensitivity tests measure only the antibiotic sensitivity of the free-floating organisms, rather than organisms in a biofilm state. As a result, a dose of antibiotics is administered to the patient, such as through a catheter, in amounts that rarely have the desired effect on the biofilm phase organisms that may reside in the catheter. The biofilm organisms may continue to shed more planktonic organisms or may go dormant and proliferate later as an apparent recurrent infection.

In order to eradicate biofilm organisms through the use of antibiotics, a laboratory must determine the concentration of antibiotic required to kill the specific genetic biofilm phase of the organism. Highly specialized equipment is required to provide the minimum biofilm eradication concentration. Moreover, the current diagnostic protocols are time consuming, and results are often not available for many days, e.g. five (5) days. This time period clearly doesn't allow for prompt treatment of infections. The delay and the well-justified fear of infection may result in the overuse of broad-spectrum antibiotics and continued unnecessary catheter removal and replacement procedures. Overuse of broad-spectrum antibiotics can result in the development of antibiotic resistant bacterial strains, which cannot be effectively treated. Unnecessary catheter removal and replacement is painful, costly and may result in trauma and damage to the tissue at the catheter insertion site.

The antibiotic resistance of biofilms, coupled with complications of antibiotic use, such as the risk of antibiotic resistant strains developing, has made antibiotic treatment an unattractive option. As a result, antibiotic use is limited to symptomatic infections and prophylactic antibiotics are not typically applied to prevent contamination. Because the biofilm can act as a selective phenotypic resistance barrier to most antibiotics, the catheter must often be removed in order to eradicate a catheter related infection. Removal and replacement of the catheter is time consuming, stressful to the patient, and complicates the medical procedure. Therefore, there are attempts to provide convenient and effective methods for killing organisms, and especially those dwelling inside of catheters, without the necessity of removing the catheter from the body.

In addition to bacterial and fungal infections, amoebic infections can be very serious and painful, as well as potentially life threatening. Several species of Acanthamoeba, for example, have been found to infect humans. Acanthamoeba are found worldwide in soil and dust, and in fresh water sources as well as in brackish water and sea water. They are frequently found in heating, venting and air conditioner units, humidifiers, dialysis units, and in contact lens paraphernalia. Acanthamoeba infections, in addition to microbial and fungal infections, may also be common in connection with other medical and dental devices, including toothbrushes, dentures and other dental appliances, and the like. Acanthamoeba infections often result from improper storage, handling and disinfection of contact lenses and other medical devices that come into contact with the human body, where they may enter the skin through a cut, wound, the nostrils, the eye, and the like.

Other areas in which infections present a problem include medical devices and materials used in connection with the eyes, such as contact lenses, scleral buckles, suture materials, intraocular lenses, and the like. In particular, there has been emphasis on discovery of methods to disinfect of ocular prosthesis, e.g. contact lenses. Bacterial biofilms may participate in ocular infections and allowing bacteria to persist on abiotic surfaces that come in contact with, or are implanted in the eye. Biofilms also may form on the biotic surfaces of the eye. “The Role of Bacterial Biofilms in Ocular Infections, DNA CELL BIOL., 21(5-6):415-20, May-June 2002. A severe form of keratitis can also be initiated by a protozoan amoeba which can contaminate lens disinfectant fluids.

In the dental field, items to be placed in a mouth, such as dental tools, dental and orthodontic devices such as retainers, bridges, dentures, and the like need to be maintained in a sterile condition, particularly during storage and prior to placement in the mouth. Otherwise, infection may be transmitted to the bloodstream and become serious.

The water supply is also prone to microbial and other types of infections. Water storage devices, as well as water supply and withdrawal conduits, often become infected. The internal surfaces of fluid bearing tubing in medical and dental offices present an environment that is suitable for microbial infection and growth and, in fact, the adherence of microbes and formation of the highly protective biofilm layer is often problematic in fluid storage and supply devices.

There is a need for improved methods and substances to prevent and destroy infections in a variety of environments. Such disinfectant solutions should have a broad range of antimicrobial properties. In particular, the solutions should be capable of penetrating biofilms to eradicate the organisms comprising the biofilms. The methods and solutions should be safe enough to be use as a preventive measure as well as in the treatment of existing infections.

N-Acetyl Cysteine (NAC) can decrease biofilm formation of a variety of bacteria and reduces the production of extra-cellular polysaccharide matrix while promoting the disruption of mature biofilm. NAC is widely used in medical practice via inhalation, oral and intravenous routes, and has an excellent safety profile. NAC is a thiol containing antioxidant that can disrupt disulfide bonds in mucus and may competitively inhibits amino acid (cysteine) utilization. NAC is available under the trade names ACC (Hexal AG), Mucomyst (Bristol-Myers Squibb), Acetadote (Cumberland Pharmaceuticals), Fluimucil and Parvolex (GSK).

Vitamin C, L-ascorbic acid, has been shown to increase the formation of nitric oxide (NO) from nitride. An increase in NO generation is expected to augment the antimicrobial activity of acidified nitrate. In addition, certain hydroxyacids, such as citric acid and lactic acid, are frequently added to foods as preservatives and are known to have antimicrobial activities.

### OBJECTS AND SUMMARY

In the following discussion, the terms “microbe” or “microbial” will be used to refer to microscopic organisms or matter, including fungal and bacterial organisms, and possibly including viral organisms, capable of infecting humans. The term “anti-microbial” will thus be used herein to refer to a material or agent that kills or otherwise inhibits the growth of fungal and/or bacterial and possibly viral organisms.

The term “disinfect” will be used to refer to the reduction, inhibition, or elimination of infectious microbes from a defined system. The term “disinfectant” will be used herein to refer to a one or more anti-microbial substances used either alone or in combination with other materials such as carriers, solvents, or the like.

The term “bactericidal activity” is used to refer to an activity that at least essentially kills an entire population of bacteria, instead of simply just reducing or inhibiting their growth. The term “fungicidal activity” is used to refer to an activity that at least essentially kills an entire population of yeast, instead of simply just reducing or inhibiting their growth. Contamination of conduits, e.g., catheters, poses serious and substantial health risks and bactericidal disinfection is a significant priority.

The term “infected system” will be used herein to refer to a defined or discrete system or environment in which one or more infectious microbes are or are likely to be present. Examples of infected systems include a physical space such as a bathroom facility or operating room, a physical object such as food or surgical tool, a biological system such as the human body, or a combination of a physical object and a biological system such as a catheter or the like arranged at least partly within a human body. Tubes and other conduits for the delivery of fluids, in industrial and healthcare settings, may also define an infected system.

A solution that consists essentially of NAC and Vitamin C in a solvent, such as water or saline, are substantially free from other active substances having material antimicrobial and/or anti-fungal activity.

The present disclosure involves disinfectant solutions comprising, or consisting essentially of, or consisting of, NAC and Vitamin C at a prescribed concentration and/or pH. The inventors have discovered, unexpectedly, that certain NAC and Vitamin C formulations provide enhanced disinfectant activities. NAC and Vitamin C formulations of the present disclosure are also highly effective in killing pathogenic biofilm organisms and are expected to reduce existing biofilms, eliminate existing biofilms, as well as prevent biofilm formation. NAC and Vitamin C formulations function as broad-spectrum anti-microbial agents, as well as fungicidal agents against many strains of pathogenic yeast. NAC and Vitamin C formulations are expected to exhibit anti-protozoan activity and also exhibit anti-amoebic activity.

The NAC and Vitamin C formulations of the present disclosure are safe for human administration and are biocompatible and non-corrosive. The disinfectant solutions of the present disclosure have numerous applications, including applications as lock and lock flush solutions for various types of catheters, use as disinfectant agents or solutions for disinfectant a range of medical, dental and veterinary devices, instruments and other objects, surfaces, and the like. They furthermore have disinfectant applications in industrial and food preparation and handling settings.

The NAC and Vitamin C formulations of the present disclosure are also have improved anticoagulant properties and are thus especially beneficial as catheter lock-flush solutions and other related uses.

The efficacy of the NAC and Vitamin C formulations of the present disclosure is superior to many disinfectant compositions conventionally used for these applications. The claimed compositions are particularly effective against biofilm organisms, which many antibiotics and biocidal agents are not. The disclosed NAC and Vitamin C formulations do not contribute to antibiotic resistance, which provides yet another important benefit.

In one embodiment, disinfectant compositions of the present disclosure have some of the following properties: anticoagulant properties; inhibitory and/or bactericidal activity against a broad spectrum of bacteria in a planktonic form; inhibitory and/or fungicidal activity against a spectrum of fungal pathogens; inhibitory and/or bactericidal activity against a broad spectrum of bacteria in a sessile form; inhibitory activity against protozoan infections; inhibitory activity against Acanthamoeba infections; safe and biocompatible, at least in modest volumes, in contact with a patient; safe and biocompatible, at least in modest volumes, in a patient's bloodstream; and safe and compatible with industrial objects and surfaces.

Methods for inhibiting the growth and proliferation of microbial populations and/or fungal pathogens are provided that comprise contacting an infected or suspected infected object, or surface, with a disinfectant composition of the present disclosure. Methods for inhibiting the growth and proliferation of protozoan populations are also provided, comprising contacting an infected or suspected infected object, or surface, with a disinfectant composition of the present disclosure.

Methods for inhibiting the growth and proliferation of amoebic populations, and for preventing amoebic infection, particularly Acanthamoeba infections, are provided, comprising contacting an object, or a surface, with a disinfectant composition of the present disclosure. Methods for substantially eradicating microbial populations are also provided and comprise contacting an infected or suspected infected object, or surface, with a disinfectant composition of the present disclosure. Methods for substantially eradicating an Acanthamoeba population are provided and comprise contacting an infected or suspected infected object, or surface, with a disinfectant composition of the present disclosure. Depending on the disinfectant composition used in the various methods, various compositions and contact time periods may be required to inhibit the formation and proliferation of various populations, and/or to substantially eradicate various populations. Suitable contact time periods for various compositions are provided in the examples and may be determined by routine experimentation.

Importantly, in most embodiments, disinfectant compositions and methods of the present disclosure do not employ traditional antibiotic agents and thus do not contribute to the development of antibiotic resistant organisms.

In one embodiment, disinfectant compositions consisting of, consisting essentially of, or comprising NAC and Vitamin C at an acidic pH are provided as disinfectant compositions of the present disclosure. Such disinfectant compositions have application as lock solutions and lock flush solutions for various types of in-dwelling access catheters, including vascular catheters used for delivery of fluids, blood products, drugs, nutrition, withdrawal of fluids or blood, dialysis, monitoring of patient conditions, and the like. Disinfectant solutions of the present disclosure may also be used as lock and lock flush solutions for urinary catheters, nasal tubes, throat tubes, and the like. The general solution parameters described below are suitable for these purposes. In one embodiment, a disinfectant solution consisting of, consisting essentially of, or comprising NAC and Vitamin C at an acidic pH is provided to maintain the patency of in-dwelling intravascular access devices. Methods for disinfecting catheters and other medical tubes, such as nasal tubes, throat tubes, and the like, are also provided and involve contacting the catheter or other medical tube with a disinfectant composition of the present disclosure.

In another embodiment, disinfectant compositions of the present disclosure consisting of, consisting essentially of, or comprising NAC and Vitamin C at an acidic pH are provided as disinfectant solutions for medical devices such as dentures and other dental and/or orthodontic and/or periodontal devices, for contact lenses and other optical devices, for medical and veterinary instruments, devices, and the like, and as disinfectant solutions for disinfectant surfaces and objects. Methods of disinfecting such devices are also provided, the methods comprising contacting a device with disinfectant compositions of the present disclosure. In general, disinfectant compositions of the present disclosure may be used as soaking solutions for dental, orthodontic and periodontal devices, including toothbrushes, and are also used as soaking solutions for contact lenses and other optical devices, and well as medical and veterinary instruments, devices, and the like. For these applications, disinfectant compositions of the present disclosure are generally formulated as solutions. Disinfectant compositions of the present disclosure are expected to be providable in a dry form which, upon introduction of a suitable solvent, forms a solution.

In yet another embodiment, disinfectant compositions of the present disclosure may be formulated for use in solutions, gels, creams and other preparations designed for topical use as disinfectant agents, wipes, antibacterial treatments, and the like. Disinfectant compositions of the present disclosure may also be used as anti-bacterial agents in connection with bandages, dressings, wound healing agents and devices, and the like.

In still another embodiment, disinfectant compositions of the present disclosure are expected to be used in industrial settings such as water storage and distribution systems, water purification, humidification and dehumidification devices, and in food preparation, handling and packaging settings to inhibit, reduce or substantially eliminate microbial populations in both planktonic and sessile forms, as well as many fungal, amoebic and planktonic populations. Industrial equipment and surfaces may be contacted or flushed with, or soaked in disinfectant compositions of the present disclosure. Time release disinfectant composition formulations may also be provided to provide treatment over time, particularly in locations that are difficult to access frequently.

### BRIEF DESCRIPTION OF THE DRAWING(S)

FIG. 1 shows the results of experiments of a NAC MIC test with P. aeruginosa. The data suggests that the MIC value for NAC is <0.25 wt %.

FIG. 2 shows the results of experiments of a NAC MIC test with S. aureus. The data suggests that the MIC value for NAC is <0.25 wt %.

FIG. 3 shows the results of experiments of a NAC MIC test with C. albicans. The data suggests that the MIC value for NAC is <1.0 wt %.

FIG. 4 shows the results of experiments of a Vitamin C MIC test with P. aeruginosa. The data suggests that the MIC value for Vitamin C is <0.25 wt %.

FIG. 5 shows the results of experiments of a Vitamin C MIC test with S. aureus. The data suggests that the MIC value for Vitamin C is <0.25 wt %.

FIG. 6 shows the results of experiments of a Vitamin C MIC test with C. albicans. The data suggests that the MIC value for VC could not be determined when 8 wt % VC was utilized as the starting concentration.

FIG. 7 shows the results of experiments of a Checkerboard Titration with P. aeruginosa. The data suggests that the FIC index=0.7 for NAC−Vitamin C Combination.

FIG. 8 shows the results of experiments of a Rate Kill Assay. The data clearly suggests the synergistic action against P. aeruginosa by a NAC+Vitamin C combination.

FIG. 9 shows the results of experiments of a Checkerboard Titration with S. aureus. The data suggests that the FIC index=0.8 for NAC−Vitamin C Combination.

FIG. 10 shows the results of experiments of a Rate Kill Assay. The data suggests that the combination is highly effective against S. aureus with NAC being the dominant component.

FIG. 11 shows the results of experiments of a Rate Kill for a NAC+Vitamin C combination against C. Albicans.

FIG. 12 shows the results of experiments of a NAC MIC test with S. aureus at a pH of 4. The data suggests that the MIC value for NAC at pH of 4 is 2.0 wt %.

FIG. 13 shows the results of experiments of a NAC MBC test with S. aureus at a pH of 4. The data suggests that the MBC value at pH of 4 is 2.0 wt %.

FIG. 14 shows the results of experiments of a NAC MIC test with S. aureus at a pH of 6. The data suggests that the MIC value for NAC at pH of 6 could not be determined with a 2.0 wt % starting point.

FIG. 15 shows the results of experiments of a Vitamin C MIC test with S. aureus at a pH of 4. The data suggests that the MIC value for Vitamin C at pH of 4 is 0.5 wt %.

FIG. 16 shows the results of experiments of a Vitamin C MBC test with S. aureus at a pH of 4. The data suggests that the MBC value for Vitamin C at pH of 4 is 1.0 wt %.

FIG. 17 shows the results of experiments of a Vitamin C MIC test with S. aureus at a pH of 6. The data suggests that the MIC value for Vitamin C at pH of 6 could not be determined with a 2.0 wt % starting point.

FIG. 18 shows the results of experiments of a Checkerboard Titration with S. aureus at a pH of 4. The data suggests that the FIC index=0.6 for NAC−Vitamin C Combination at a pH of 4.

FIG. 19 shows the results of experiments (raw data) of a Prothrombin Time (PT) Assay.

FIG. 20 shows the results of experiments (processed data) of a Prothrombin Time (PT) Assay.

FIG. 21 shows the graph of the International Normalized Ratio (INR) for NAC from a Prothrombin Time (PT) Assay.

FIG. 22 shows the graph of the International Normalized Ratio (INR) for Vitamin C from a Prothrombin Time (PT) Assay.

FIG. 23 shows the graph of the International Normalized Ratio (INR) for combined NAC and Vitamin C formulations from a Prothrombin Time (PT) Assay.

### DETAILED DESCRIPTION

Disinfectant compositions of the present disclosure may comprise concentrations of NAC and Vitamin C at an acidic pH. NAC and Vitamin C may be used in compositions with water as the solvent.

NAC is also known as L-alpha-acetamido-beta-mercaptopropionic acid, acetein, acetylcysteine, N-acetylcysteine, N-acetyl-N-cysteine, Nacetyl-3-mercaptoalanine, airbron, broncholysin, fluimucetin, fluimucil, flumicil, inspir, mercapturic acid, mucolyticum, mucolyticum lappe, mucolytikum lappe, mucomyst, mucosolvin, NAC, NAC-TB, NSC 111180, parvolex, respaire. Some physical properties of NAC are:
Appearance: White to white with light yellow cast powder
Melting Point: 109-110° C.
Molecular formula: C5H9NO3S
Formula weight: 163.2 (anhydrous)
pKa: 9.5 at 30° C.
Optical rotation: +5° (c=3% in water)
Purity: Not less than 99% (TLC)
   N-acetyl-L-cysteine (NAC) is soluble 1 g in 8 mL of water and 1 g in 2 mL of ethanol. It is practically insoluble in chloroform and ether. Aqueous solutions of cysteine, and thus also likely applicable to N-acetyl-L-cysteine, oxidize to cystine on contact with air at neutral or alkaline pH. Oxidation is accelerated by traces of heavy metals, especially copper and iron; relatively stable in acid.

Some properties of vitamin C are:
Molecular Formula: C6H8O6
Molecular Weight: 176.1
CAS Number: 50-81-7
pKa: 4.17 and 11.57
Melting Point: 190-192° C.
Extinction Coefficient EmM=7.0 (265 nm, water), 7.5 (245 nm, acid)
Rotation: +20.5° to +21.5° (100 mg/ml H2O, 25° C.)
   This product is soluble in water (50 mg/ml), yielding a clear solution. Aqueous solutions are stable only in the absence of oxygen. Aqueous solutions are most stable at pH 5-6, and very unstable at alkaline pH. Degradation is markedly increased in the presence of transition metal ions, especially Cu²⁺ and Fe³⁺. The first stage of oxidation of L-ascorbic acid to dehydroascrobic acid is reversible and the biological activity is retained. Further oxidation to 2,3-diketogulonic acid is not reversible and the activity is lost.

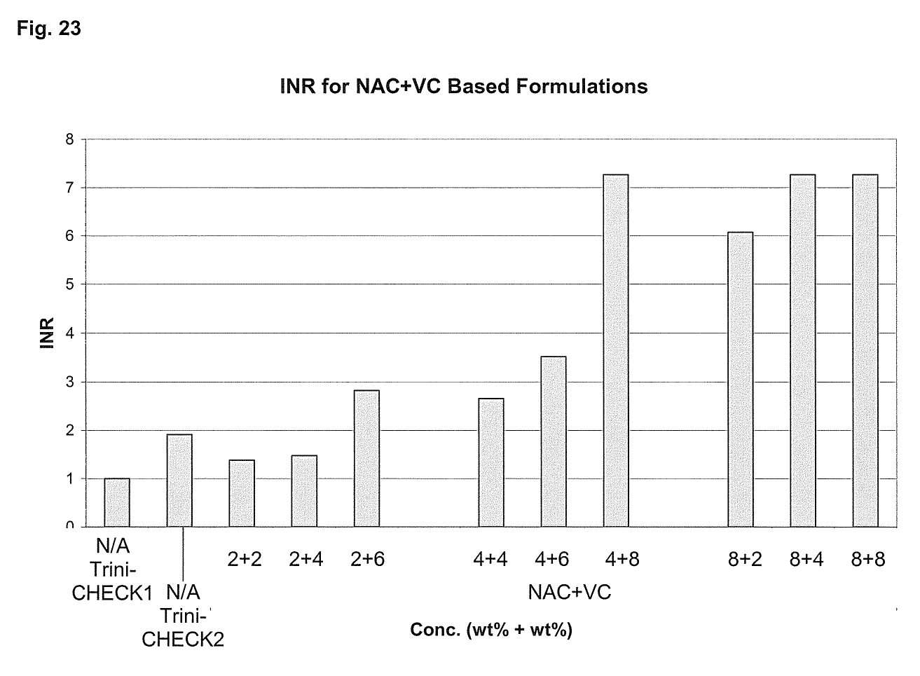

NAC by itself has been shown to have some anti-coagulant effect. See, e.g., Niemi, T. T., et al., The effect of N-acetylcysteine on blood coagulation and platelet function in patients undergoing open repair of abdominal aortic aneurysm, Blood Coagul Fibrinolysis, 2006, 17(1): p. 29-34 and Pol, S. and P. Lebray, N-acetylcysteine for paracetamol poisoning: effect on prothrombin. The Lancet, 2002, 360(9340): p. 1115. Vitamin C has not been identified as an effective anticoagulant when compared with heparin. See, e.g., Rabe, C., et al., Keeping central venous lines open: a prospective comparison of heparin, vitamin C and sodium chloride sealing solutions in medical patients, Intensive Care Med, 2002, 28(8): p. 1172-6. The combination of NAC and Vitamin C has an anti-coagulant effect. Moreover, the combination of NAC and Vitamin C shows an unexpected synergism (See FIG. 23) as the combination of NAC and Vitamin C has an unexpected improvement in anti-coagulant effect as compared to either NAC or Vitamin C alone.

Embodiments of the disclosed composition may comprise at least 0.01% NAC, by weight per volume solution (w/v) and up to 12% (w/v) NAC. Embodiments comprising at least 0.1% (w/v) NAC and less than 2.5% (w/v) NAC are preferred for many applications, compositions comprising at least 0.1% (w/v) NAC and less than 1.0% (w/v) NAC are also preferred for certain applications and compositions comprising about 0.25% (w/v) NAC are especially preferred.

Embodiments of the disclosed composition may comprise at least 0.01% Vitamin C, by weight per volume solution (w/v) and up to 12% (w/v) Vitamin C. Embodiments comprising at least 0.1% (w/v) Vitamin C and less than 2.5% (w/v) Vitamin C are preferred for many applications, compositions comprising at least 0.1% (w/v) Vitamin C and less than 1.0% (w/v) Vitamin C are also preferred for certain applications and compositions comprising about 0.25% (w/v) Vitamin C are especially preferred.

Embodiments of the disclosed composition may comprise between 0 and 25% (v/v) ethanol and water. Other embodiments of the disclosed composition may comprise between 0 and 20% (v/v) ethanol and water, between 0 and 15% (v/v) ethanol and water, or between 0 and 10% (v/v) ethanol and water.

The desired NAC and Vitamin C concentrations for various applications may depend on the type of infection being treated and, to some degree, on the solvent used for disinfectant compositions. When aqueous solvents comprising ethanol are used, for example, the concentrations of NAC and Vitamin C required to provide the desired level of activity may be reduced compared to the NAC and Vitamin C concentrations used in compositions having water as the solvent. “Effective” concentrations of NAC and Vitamin C in disinfectant compositions of the present disclosure for inhibitory, bactericidal, fungicidal, biofilm eradication and other purposes may be determined by routine experimentation.

In certain embodiments, disinfectant compositions of the present disclosure comprise, or consist essentially of, or consist of, NAC and Vitamin C in solution at an acidic pH, preferably at a pH of < or ≦6.0, or at a pH< or ≦5.0, or at a pH< or ≦4.5, or at a pH< or ≦4.0.

Disinfectant compositions comprising, or consisting essentially of, or consisting of NAC and Vitamin C have different “effective” pH ranges. “Effective” pH ranges for desired NAC and Vitamin C in disinfectant compositions of the present disclosure for inhibitory, bactericidal, fungicidal, biofilm eradication and other purposes may be determined by routine experimentation.

In some embodiments, disinfectant compositions of the present disclosure consist of NAC and Vitamin C, as described above, and disinfectant solutions consist of NAC and Vitamin C dissolved in a solvent, generally an aqueous solvent such as water or saline. In other embodiments, disinfectant compositions of the present disclosure consist essentially of NAC and Vitamin C, as described above, generally in an aqueous solvent such as water or saline.

In some embodiments, disinfectant compositions of the present disclosure comprise NAC and Vitamin C having specified concentrations, at specified pH ranges, and may contain materials, including active components, in addition to the NAC and Vitamin C described above. Other antimicrobial or biocidal components may be incorporated in disinfectant compositions of the present disclosure comprising NAC and Vitamin C, although the use of traditional antibiotics and biocidal agents is generally discouraged as a result of the potential dire consequences of the development of antibiotic- and biocidal-resistant organisms. In some embodiments, disinfectant compositions of the present disclosure comprising NAC and Vitamin C having specified concentration(s), at specified pH ranges, are substantially free from other active substances having substantial antimicrobial and/or anti-fungal activity.

Other active and inactive components may also be incorporated in disinfectant compositions of the present disclosure comprising NAC and Vitamin C, preferably provided that they don't deleteriously affect the activity and/or stability of the NAC and Vitamin C. Proteolytic agents may be incorporated in disinfectant compositions for some applications. Disinfectant compositions formulated for topical application have various creams, emollients, skin care compositions such as aloe vera, and the like, for example. Disinfectant compositions of the present disclosure provided in a solution formulation may also comprise other active and inactive components, preferably provided they don't interfere, deleteriously, with the activity and/or stability of the NAC and Vitamin C.

The compositions of the present disclosure may be used in a solution or a dry form. In solution, the NAC and Vitamin C are preferably dissolved in a solvent, which may comprise an aqueous solution, such as water or saline, or another biocompatible solution in which the NAC and Vitamin C are soluble. Other solvents, including alcohol solutions, may also be used. In one embodiment, NAC and Vitamin C compositions of the present disclosure are formulated in a mixture of water and ethanol. Such solutions are highly efficacious and may be prepared by making a concentrated NAC and Vitamin C stock solution in water and then introducing the desired concentration of ethanol. Ethanol concentrations of from more than about 0.5% and less than about 10%, v/v, provide effective disinfectant compositions. In some embodiments, bio-compatible non-aqueous solvents may also be employed, provided the NAC and Vitamin C can be solubilized and remain in solution during storage and use.

NAC and Vitamin C solutions of the present disclosure are preferably provided in a sterile and non-pyrogenic form and may be packaged in any convenient fashion. In some embodiments, disinfectant NAC and Vitamin C compositions of the present disclosure may be provided in connection with or as part of a medical device, such as in a pre-filled syringe or another medical device. The compositions may be prepared under sterile, aseptic conditions, or they may be sterilized following preparation and/or packaging using any of a variety of suitable sterilization techniques. Single use vials, syringes or containers of NAC and Vitamin C solutions may be provided. Multiple use vials, syringes or containers may also be provided. Systems of the present disclosure include such vials, syringes or containers containing the NAC and Vitamin C solutions of the present disclosure.

The compositions of the present disclosure may also be provided in a substantially “dry” form, such as a substantially dry coating on a surface of tubing, or a conduit, or a medical or industrial device such as a catheter or conduit, or a container, or the like. Dry forms of the disinfectant compositions of the present disclosure may include hydrophilic polymers such as PVP, which tend absorb water and provide lubricity, surfactants to enhance solubility and/or bulking and buffering agents to provide thermal as well as pH stability. Such substantially dry forms of NAC and Vitamin C compositions of the present disclosure may be provided in a powder or lyophilized form that may be reconstituted to form a solution with the addition of a solvent. Substantially dry forms of NAC and Vitamin C compositions may alternatively be provided as a coating, or may be incorporated in a gel or another type of carrier, or encapsulated or otherwise packaged and provided on a surface as a coating or in a container. Such substantially dry forms of NAC and Vitamin C compositions of the present disclosure are formulated such that in the presence of a solution, the substantially dry composition forms an NAC and Vitamin C solution having the composition and properties described above. In certain embodiments, different encapsulation or storage techniques may be employed such that effective time release of the NAC and Vitamin C is accomplished upon extended exposure to solutions. In this embodiment, the substantially dry NAC and Vitamin C solutions may provide disinfectant activity over an extended period of time and/or upon multiple exposures to solutions.

Compositions comprising NAC have a well established safety profile in connection with medical usage and administration to humans. For example, a dose of 1200 mg/day has been shown to be safe for administration to humans. See, e.g., High Dose N-Acetylcysteine in Patients With Exacerbations of Chronic Obstructive Pulmonary Disease, R. Zuin; A. Palamidese; R. Negrin; L. Catozzo; A. Scarda; M. Balbinot; Clin Drug Invest. 2005; 25(6):401-408. This dose is well tolerated. NAC is also present, in combination with other components, in many solutions used in medical and human health applications, and has been established as safe for human use, both in vitro and in vivo. NAC is readily available at a reasonable cost, and is stable over time in solution.

Compositions comprising Vitamin C have a well established safety profile in connection with medical usage and administration to humans. For example, a dose of anywhere between 60 mg-18000 mg/day for an adult human being as been shown to be safe. See, e.g., US Recommended Dietary Allowance (RDA). Retrieved on 2007-02-19 and Pauling, Linus (1986). How to Live Longer and Feel Better. W. H. Freeman and Company. ISBN 0-380-70289-4. This dose is well tolerated. Vitamin C is also present, in combination with other components, in many solutions used in medical and human health applications, and has been established as safe for human use, both in vitro and in vivo. Vitamin C is readily available at a reasonable cost, and is stable over time in solution.

Formulation and production of disinfectant compositions of the present disclosure are generally straightforward. In one embodiment, desired disinfectant compositions of the present disclosure are formulated by dissolving NAC and Vitamin C in an aqueous solvent, such as purified water, to the desired concentration and adjusting the pH of the solution to the desired pH. In alternative embodiments, desired disinfectant compositions of the present disclosure are formulated by dissolving NAC and Vitamin C in a solvent in which the NAC and Vitamin C are soluble to provide a concentrated, solubilized solution, and additional solvents or components may then be added, or the solubilized composition may be formulated in a form other than a solution, such as a topical preparation. The disinfectant solution may then be sterilized using conventional means, such as filtration and/or ultrafiltration, and other means. The osmolarity range for NAC and Vitamin C solutions may be from 116 to 500 mOsm/Kg, 240-500 mOsm/Kg, or preferably from 300-420 mOsm/Kg. A 4 wt % (w/v) NAC solution has a osmolarity of 237 mOsm/Kg, a 4 wt % (w/v) Vitamin C solution has a osmolarity of 223 mOsm/Kg, and a solution with a combination of 4 wt % (w/v) NAC and 4 wt % (w/v) Vitamin C has an osmolarity of 435 mOsm/Kg. The solutions are preferably formulated using USP materials.

Disinfectant compositions of the present disclosure comprising, or consisting essentially of, or consisting of, NAC and Vitamin C as described above are also useful for many other applications. NAC and Vitamin C solutions may be used as disinfectant solutions for soaking, or rinsing, or contacting medical, dental and veterinary surfaces and objects. NAC and Vitamin C solutions of the present disclosure may be used, for example, for storing and/or disinfectant contact lenses and other optical devices; for storing and/or disinfectant dental devices such as dentures, bridges, retainers, tooth brushes, and the like, and for storing and/or disinfectant medical and dental and veterinary devices and instruments. In these applications, the devices or surfaces may be contacted with NAC and Vitamin C solutions of the present disclosure for a time sufficient to substantially eliminate microbial and/or fungicidal infections, or devices and surfaces may be soaked in NAC and Vitamin C solutions for a desired time period. NAC and Vitamin C compositions of the present disclosure may additionally be used to disinfect water and other fluid supply lines. Disinfectant of fluid supply lines may be accomplished by intermittently flushing the lines with NAC and Vitamin C compositions of the present disclosure. Similarly, NAC and Vitamin C compositions of the present disclosure may be used to eradicate biofilms and microbial (including some virus and protozoa) and fungal populations in water supply and storage devices.

Conduits are expected to be treated with NAC and Vitamin C solutions as a preventative disinfectant or as treatment following potential fungal or bacterial infection.

The treatment of conduits can include locking, flushing, coating, or aerosol doses of the NAC and Vitamin C solution. Examples of conduits that may be treated using the NAC and Vitamin C solution include water lines in dental or medical offices, lines carrying sterile fluids, catheters or ports that carry blood and/or other fluids into and out of the body, industrial water supply lines which develop large biofilm populations which effect the efficient flow of fluids as well as contaminating the fluids passing through the line, and airway support devices. Other examples include consumption such as drink dispensers and food packaging. Conduits treated by the NAC and Vitamin C solution are typically made of plastic, but the principles of the present disclosure may be applied to conduit device made of any material such as metal that delivers or carries fluid.

A NAC and Vitamin C solution can be used in treatment of topical infections, including but not limited to skin, ear, anal, mouth, and vulvo/vaginal sites.

A NAC and Vitamin C solution can be used in as an effective disinfectant for surfaces and equipment in industrial, medical, and household applications. A typical infected system would include the walls, floors, and commode in a lavatory. The delivery system will typically comprise a solvent and tools that allow flushing, locking, wiping, soaking, fogging, or coating of the surface defining the infected system.

A NAC and Vitamin C solution can be used as an effective decontamination disinfectant for medical instruments and devices, dental (both consumer and professional) instruments and devices, and/or veterinary instruments and devices. A typical example would be a soak for disinfecting toothbrushes.

A NAC and Vitamin C solution can be used as an effective disinfectant solution for optical contact lenses.

A NAC and Vitamin C solution can be used as a treatment for catheters defining an infected system. The NAC and Vitamin C solution may inhibit microbe colonization by treating the catheter with the solution at the prescribed concentration using a liquid lock prior to and in between infusions and/or by surface coating of catheter devices. A further application is the treatment of colonized or infected catheters by use of a liquid lock containing the NAC and Vitamin C solution in the preferred concentration and pH.

Typically, the NAC and Vitamin C solution, when used to treat catheters, are dissolved in water as a carrier, although other carriers may be used. Substances such as thrombolytics, sodium, alcohol, or reagents may also be added to the basic water/NAC and Vitamin C solution.

### MIC Experiments

The minimum concentration of a composition required to inhibit growth is known as the minimum inhibitory concentration (MIC). In order to determine MIC a National Committee on Clinical Laboratory Standards (NCCLS) micro-dilution procedure was followed. According to the procedure each formulation must be exposed to 6 log concentration or the highest achievable concentration of organism. In the current protocol 100 μL of MHB was mixed with 90 μL of formulation and 10 μL of log 8 organism or the highest achievable concentration. The concentration of the formulation was adjusted to obtain the required concentration in the final solution. The mixture was incubated at 37 degree C. for 16-24 hrs. After 16-24 hours the absorbance value was read at 600 nm. The obtained data was corrected by subtracting the appropriate blanks. Finally, the wells having an absorbance≧0.1 were marked + and <0.1 were marked −. The + symbol indicated growth while − symbol indicates no growth. The positive growth controls must have a corrective absorbance value of >0.5 and negative controls must have a corrected absorbance value of <0.1. In cases where the positive growth controls corrected absorbance is lower than 0.5, an alternate rule is utilized which is “absorbance<than 20% of positive growth control is marked as − growth, while absorbance≧than 20% of positive growth control is marked as + growth”.

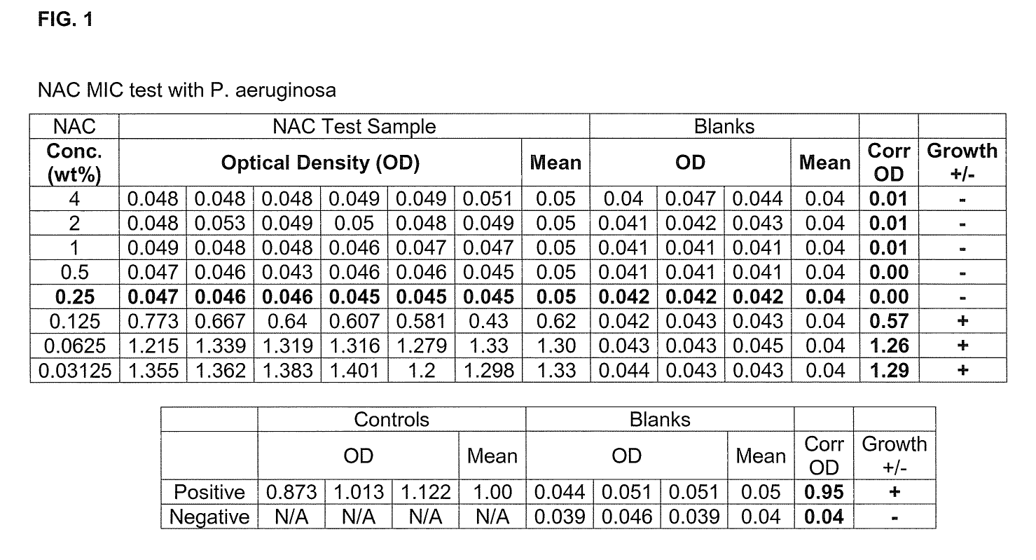

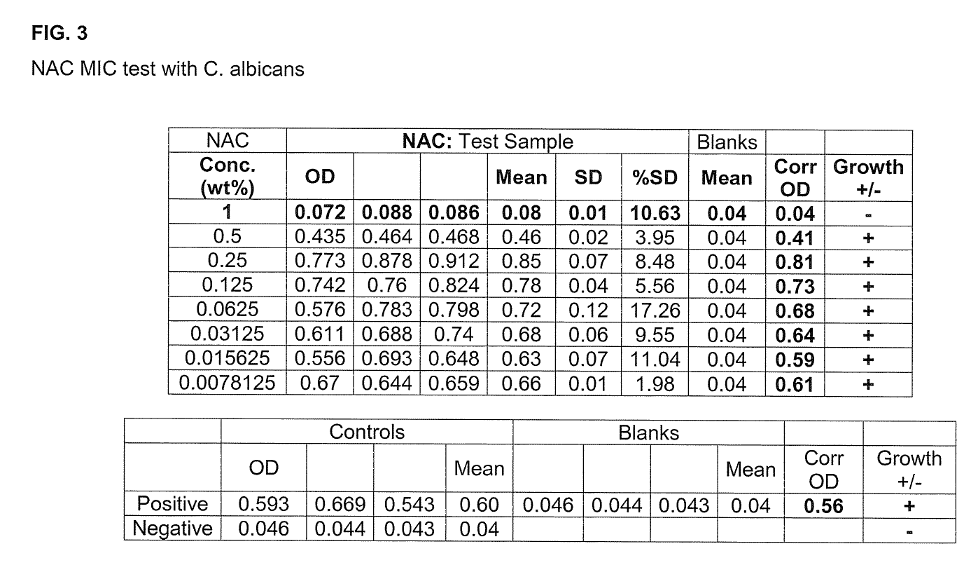

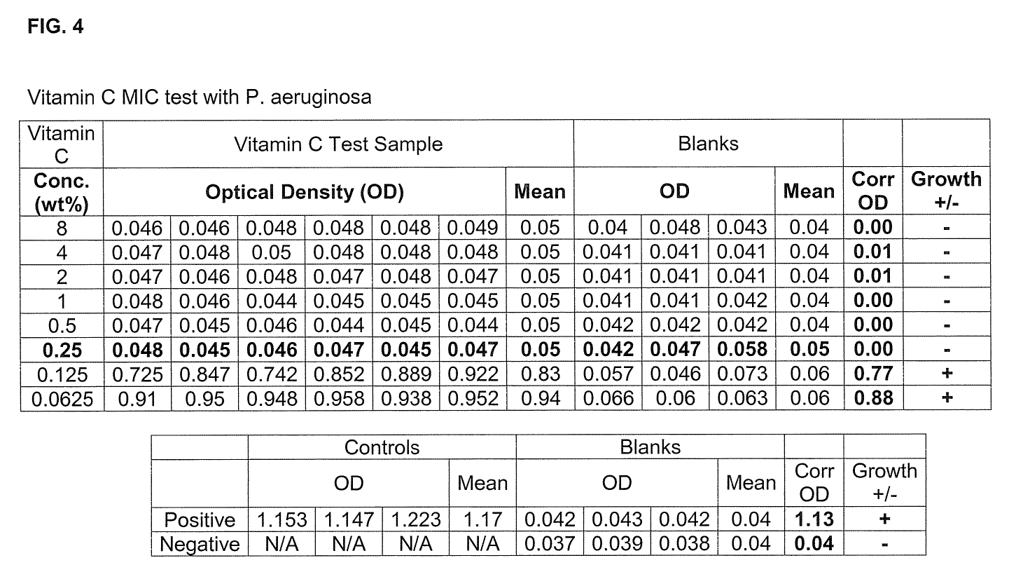

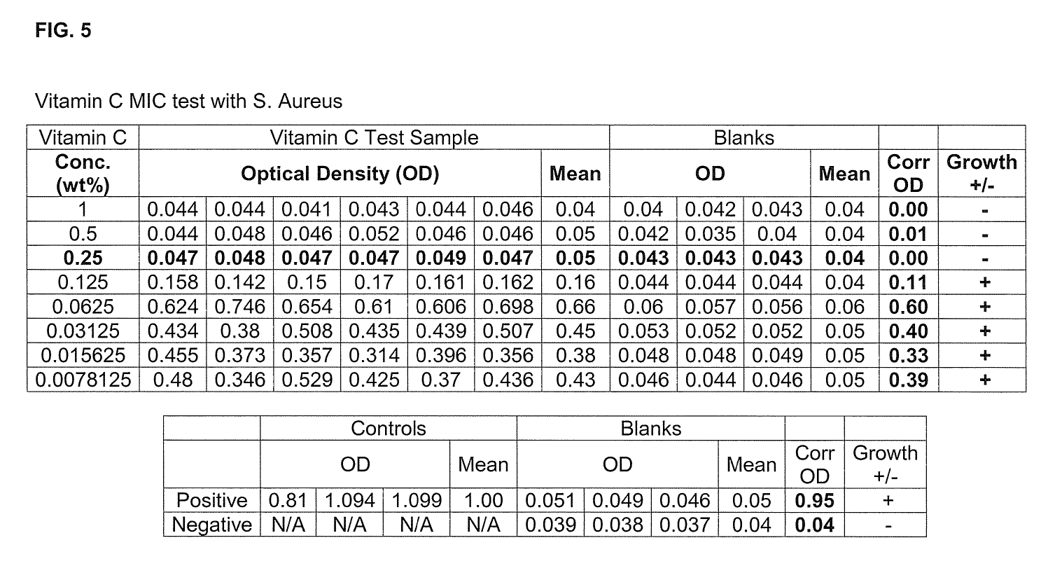

Staphylococcus aureus (organism # 25923), Pseudomonas aeruginosa (Organism # 27853) and Candida Albicans (Organism # 10231) was obtained from ATCC. L-Ascorbic Acid (Vitamin C) was used (Fisher Scientific, Catalogue # A61-25, Lot # 066251). N-acetyl cysteine (NAC) was used (Acros, Catalogue # 160280250, Lot # A0229576). A 8 wt % NAC solution in water was prepared. A 16 wt % Vitamin C solution in water was prepared. These solutions were then serially diluted as necessary to obtain the required concentrations. A minimum concentration of NAC and Vitamin C that inhibited the growth of Staphylococcus aureus and P. aeruginosa was found. In addition, a minimum concentration of NAC that inhibited the growth of Candida Albicans was found, but a minimum concentration of Vitamin C that inhibited the growth of Candida Albicans was not able to be determined (per experiments conducted it was >8 wt %). As per experiments conducted, NAC has a MIC of <0.25% (w/v) for S. aureus, Vitamin C has a MIC of <0.25% (w/v) for S. aureus, NAC has a MIC of <0.25% (w/v) for P. aeruginosa, Vitamin C has a MIC of <0.25% (w/v) for P. aeruginosa and NAC has a MIC of <1.0% (w/v) for C. albicans. See FIGS. 1-6 for MIC results.

### Synergism Experiments

Experiments were conducted to show an unexpected synergism of the disinfectant activity of a composition that includes both NAC and Vitamin C.

A first experiment conducted was a screening experiment using checkerboard titration to assess if the combinations fall within a range having an FIC index value of <1. The method used was a NCCLS micro-dilution procedure.

A second experiment conducted was a “rate of kill” assay. A rate of kill assay can confirm whether combinations are synergistic or not. In this assay the formulations are first exposed to organisms for a desired time (the current formulations readings were taken at 0, 1, 2, 3 and 24 hrs). Then a sample of the organisms and formulation mixture is serially diluted and plated to assess the log recovery. The organisms are allowed to grow and are checked for growth/log recovery after 24 hrs. The log recovery values obtained for individual components were compared with the combinations. Any combinations having ≧2 log reduction when compared with the most active compound used in the combination at any time point tested were labeled as synergistic (Comparison of methods for assessing synergic antibiotic interactions, International journal of antimicrobial agents, 15 (2000) 125-129).

According to the first and second experiments described above, experiments were conducted to investigate the effect of Vitamin C on the antimicrobial activity of NAC. L-Ascorbic Acid (Vitamin C) was used (Fisher Scientific, Catalogue # A61-25, Lot # 066251). N-acetyl cysteine (NAC) was used (Acros, Catalogue # 160280250, Lot # A0229576).

### Checkerboard Titration Experiment

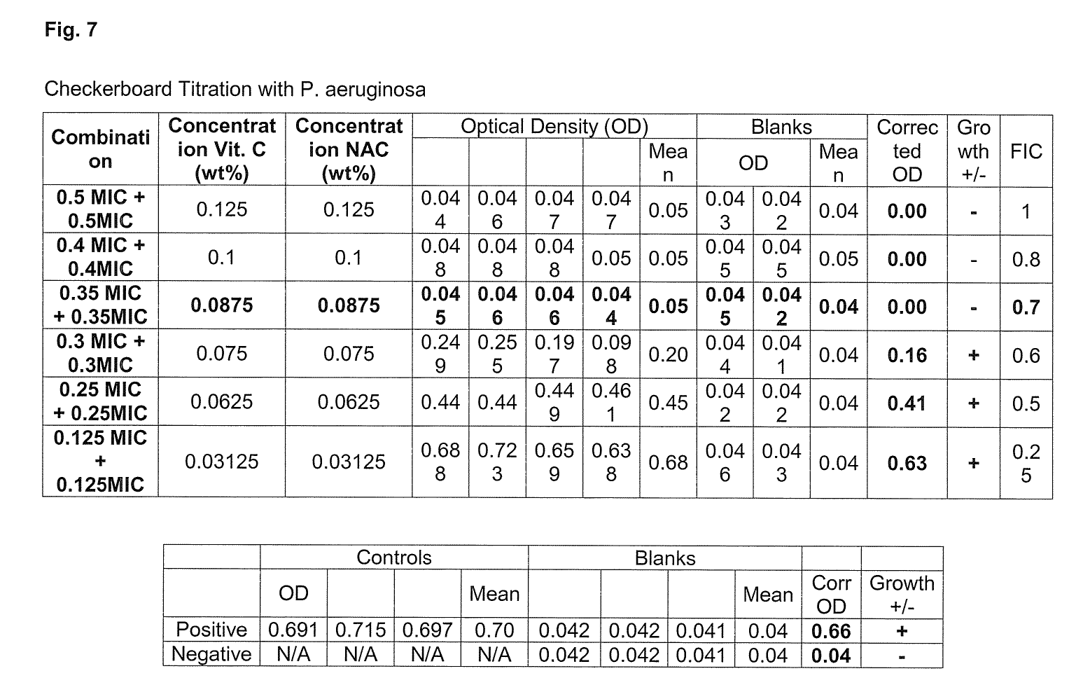

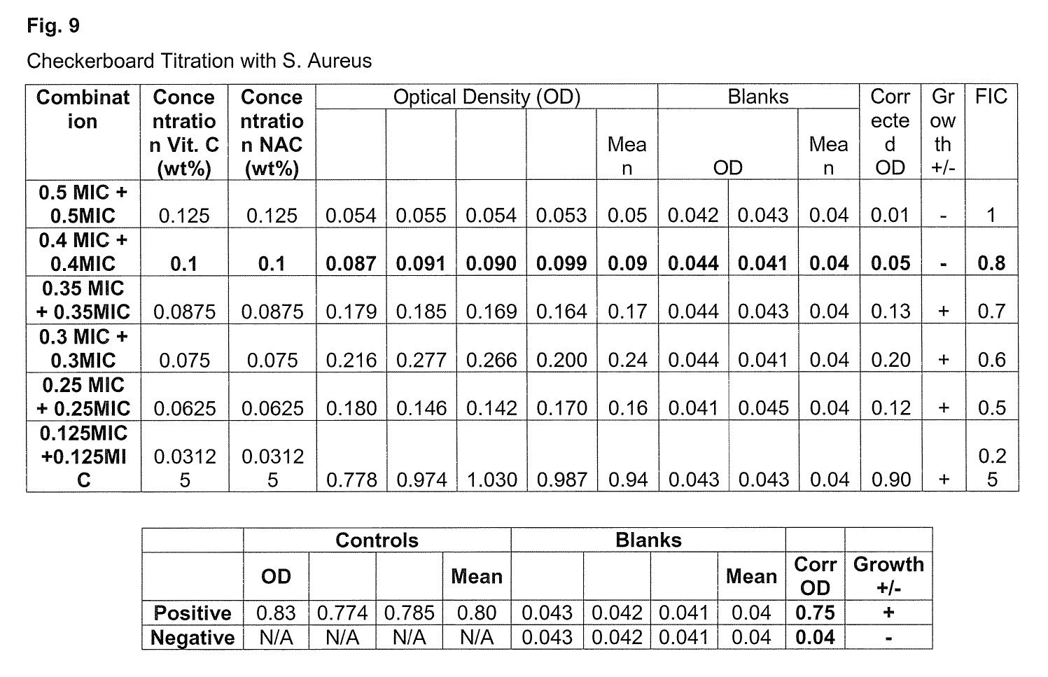

The Checkerboard Titration method was used to assess the interactions between NAC and Vitamin C. The Checkerboard Titration method is a frequently used technique where, for example, each agent (NAC and Vitamin C) was tested at multiple dilutions lower than the MIC. During this experiment, NAC and Vitamin C were tested in the combinations to assess if the combinations have an FIC index of <1. The following concentrations were tested (for S. aureus see FIG. 9 and for P. aeruginosa see FIG. 7 for combinations at a pH<4):

| | Concentration | Concentration |
|---|---|---|
| Combination | Vit. C (wt %) | NAC (wt %) |
| 0.5 MIC + | 0.125 | 0.125 |
| 0.5 MIC | | |
| 0.4 MIC + | 0.1 | 0.1 |
| 0.4 MIC | | |
| 0.35 MIC + | 0.0875 | 0.0875 |
| 0.35 MIC | | |
| 0.3 MIC + | 0.075 | 0.075 |
| 0.3 MIC | | |
| 0.25 MIC + | 0.0625 | 0.0625 |
| 0.25 MIC | | |
| 0.125 MIC + | 0.03125 | 0.03125 |
| 0.125 MIC | | |

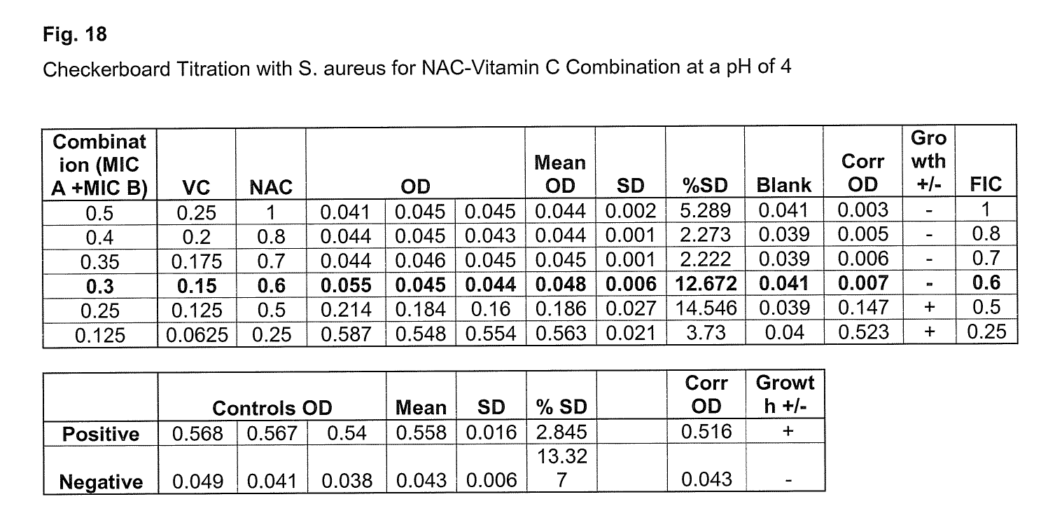

The following concentrations were tested against S. aureus (see FIG. 18) for combinations at a pH of 4:

| | Concentration | Concentration |
|---|---|---|
| Combination | Vit. C (wt %) | NAC (wt %) |
| 0.5 MIC + | 0.25 | 1 |
| 0.5 MIC | | |
| 0.4 MIC + | 0.2 | 0.8 |
| 0.4 MIC | | |
| 0.35 MIC + | 0.175 | 0.7 |
| 0.35 MIC | | |
| 0.3 MIC + | 0.15 | 0.6 |
| 0.3 MIC | | |
| 0.25 MIC + | 0.125 | 0.5 |
| 0.25 MIC | | |
| 0.125 MIC + | 0.0625 | 0.25 |
| 0.125 MIC | | |

Fraction Inhibitory Concentration (FIC) is defined as the MIC of the compound in combination divided by the MIC of the compound alone. If the FIC index is ≦0.5, the combination is interpreted to be synergistic; <1 but >0.5—as partially synergistic; =1 as additive; >1 but <4 as indifferent; and ≧4 as antagonistic. In order to calculate the FIC index the following calculations are performed for compounds A and B:

FIC-A=(MIC of A in combination)/(MIC of A alone)

FIC-B=(MIC of B in combination)/(MIC of B alone)

FIC-combination=FIC-A+FIC-B

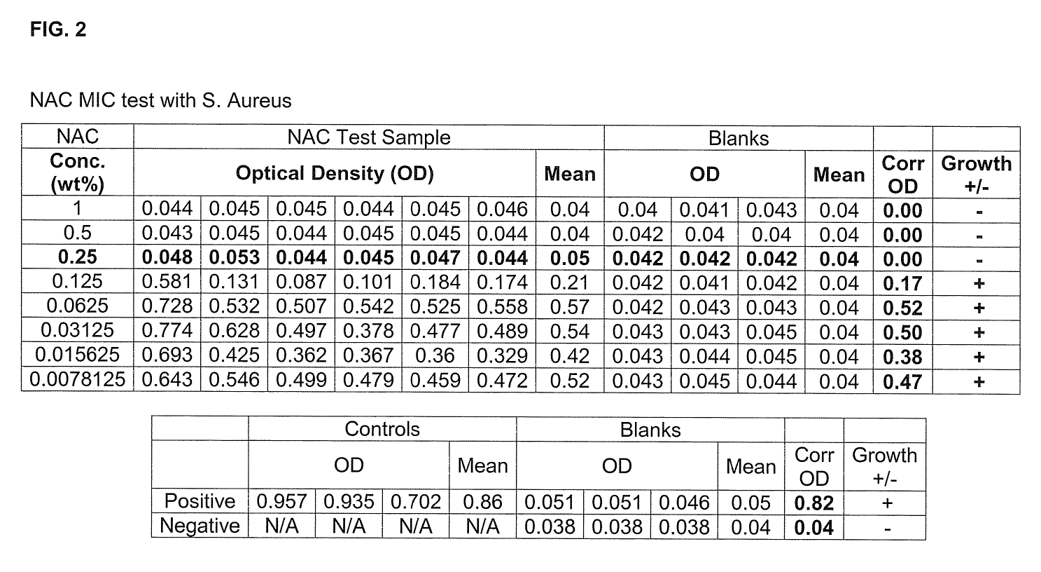

The MIC-NAC (MIC of NAC in combination with Vitamin C), a minimum concentration of NAC, while in combination with Vitamin C, that inhibited the growth of S. aureus as well as P. aeruginosa in MHB was found. In order to determine the MIC-VC (MIC of Vitamin C in combination with NAC), a minimum concentration of Vitamin C, while in combination with NAC, that inhibited the growth of S. aureus as well as P. aeruginosa in MHB was found. As per experiments conducted above, the MIC-NAC is 0.25% (w/v) for S. aureus and the MIC-VC is 0.25% (w/v) for Staphylococcus aureus. See FIGS. 2, 5 and 9 for results. As per experiments conducted above, the MIC-NAC is 0.25% (w/v) for P. aeruginosa and the MIC-VC is 0.25% (w/v) for P. aeruginosa. See FIGS. 1, 4 and 7 for results.

Thus against S. Aureus, the FIC-NAC is 0.1/0.25, which equals 0.4. The FIC-VC is 0.1/0.25, which equals 0.4. Thus, the FIC-combination is 0.4+0.4, which equals 0.80. While against P. aeruginosa, the FIC-NAC is 0.0875/0.25, which equals 0.35. The FIC-VC is 0.0875/0.25, which equals 0.35. Thus, the FIC-combination is 0.35+0.35, which equals 0.70. Accordingly, the combination of NAC and Vitamin C unexpectedly has partial synergistic results. That is, embodiments of the combination of NAC and Vitamin C provides results that are, unexpectedly, greater than the total effects of each agent operating by itself. This partial synergistic effect of embodiments of the combination of NAC and Vitamin C may provide enhanced activity against biofilms. Without being bound to theory, it is suspected that NAC will degrade the biofilm, thus making the sessile microorganisms planktonic, allowing Vitamin C to act against the more susceptible planktonic microorganisms.

The interaction between NAC & VC against C. Albicans data via Checkerboard Method is not available. This is due to the fact that the MIC for vitamin C against C. Albicans could not be obtained when 8 wt % Vitamin C was utilized as the starting concentration.

### Rate Kill Assay

As discussed above, NAC has a MIC of <0.25% (w/v) for S. aureus and P. aeruginosa, and Vitamin C has a MIC of <0.25% (w/v) for S. aureus and P. aeruginosa. Accordingly, the following solutions were prepared:

| Composition | wt % (w/v) | MIC |
|---|---|---|
| NAC | 0.125 wt % | 0.5 |
| Vitamin C | 0.125 wt % | 0.5 |
| NAC | 0.0625 wt % | 0.25 |
| Vitamin C | 0.0625 wt % | 0.25 |
| NAC + Vitamin C | 0.125 wt % + 0.125 wt % | 0.5 + 0.5 |
| NAC + Vitamin C | 0.0625 wt % + 0.0625 wt % | 0.25 + 0.25 |

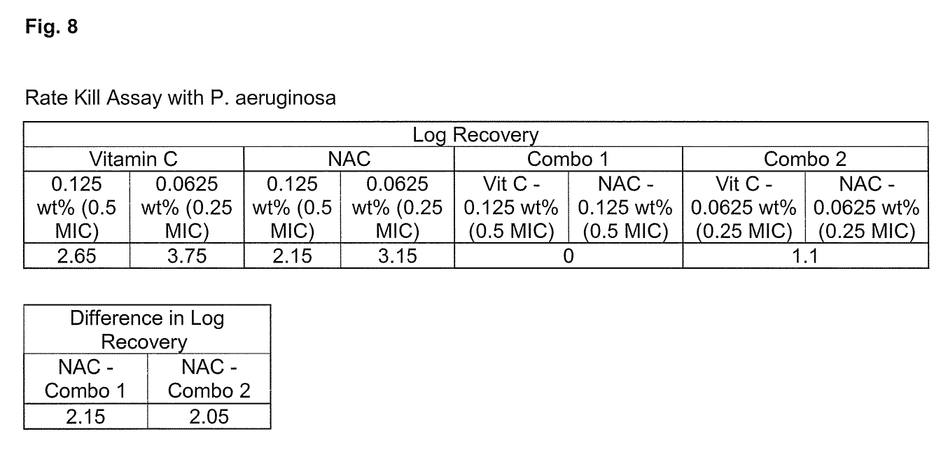

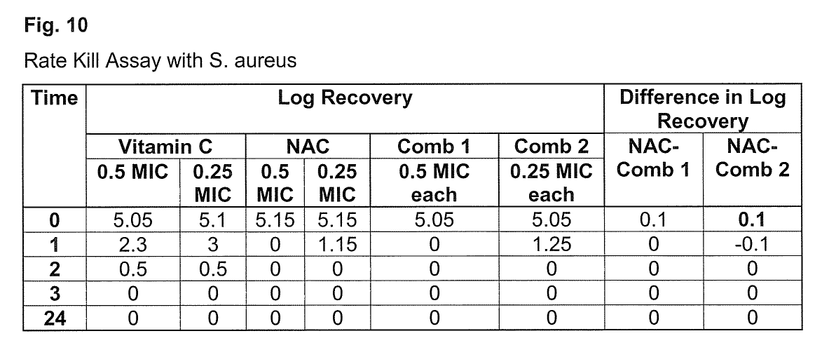

Each solution was then combined with P. aeruginosa, and separately with S. Aureus. The log recovery of the P. aeruginosa as well as S. Aureus was measured after 24 hours. For P. aeruginosa the difference in log recovery for the 0.5 MIC concentrations was 2.15 and the difference in log recovery for the 0.125 MIC concentrations was 2.05. See FIG. 8 for results. For S. Aureus the difference in log recovery for the 0.5 MIC concentrations was 0.1 and the difference in log recovery for the 0.125 MIC concentrations was 0.1. See FIG. 10 for results. Accordingly, the data shows that against P. Aeruginosa NAC and Vitamin C solutions are synergistic while against S. Aureus the combination is highly effective. That is, embodiments of the combination of NAC and Vitamin C provides results that are, unexpectedly, greater than the total effects of each agent operating by itself. This effect of embodiments of the combination of NAC and Vitamin C is expected to provide enhanced activity against biofilms. Without being bound to theory, it is suspected that NAC will degrade the biofilm, thus making the sessile microorganisms planktonic, allowing Vitamin C to act against the more susceptible planktonic microorganisms.

The synergistic effect (rate kill assay) and partial synergistic effect (checkerboard titration) provides significant, practical advantages for uses of embodiments of the combination of NAC and Vitamin C. As noted, biofilms are a significant problem in a variety of fields. The biofilm protective substance, often referred to as extra-cellular polymeric substance (EPS), polysaccharide covering or glycocalyx, provides a protection to biofilms that are difficult to inhibit or eradicate. A solution that can inhibit or eradicate a biofilm is an important alternative. The proper use of antibiotics to eradicate a biofilm is costly, time consuming and may result in the development of antibiotic resistant bacterial strains, which cannot be effectively treated. Thus, embodiments of the present disclosure should prevent the overuse of broad-spectrum antibiotics and continued unnecessary catheter removal and replacement procedures.

### Rate Kill—Fungus Test

A Rate Kill experiment was conducted to investigate the effect of Vitamin C on the antifungal activity of NAC. L-Ascorbic Acid (Vitamin C) was used (Fisher Scientific, Catalogue # A61-100, Lot # 074355). N-acetyl cysteine (NAC) was used (Acros, Catalogue # 16028-0500, Lot # B0122404). A C. Albicans (ATCC-10231) organism was used.

The following solutions were prepared:

| Composition | wt % (w/v) | MIC | pH |
|---|---|---|---|
| Vitamin C | 8 wt % | N/A | 2.32 |
| Vitamin C | 4 wt % | N/A | 2.61 |
| Vitamin C | 2 wt % | N/A | 2.72 |
| 0.5 MIC-NAC | 0.5 wt % | 0.50 | 2.58 |
| 0.25 MIC-NAC | 0.25 wt % | 0.25 | 2.70 |
| 0.125 MIC-NAC | 0.125 wt % | 0.125 | 2.83 |

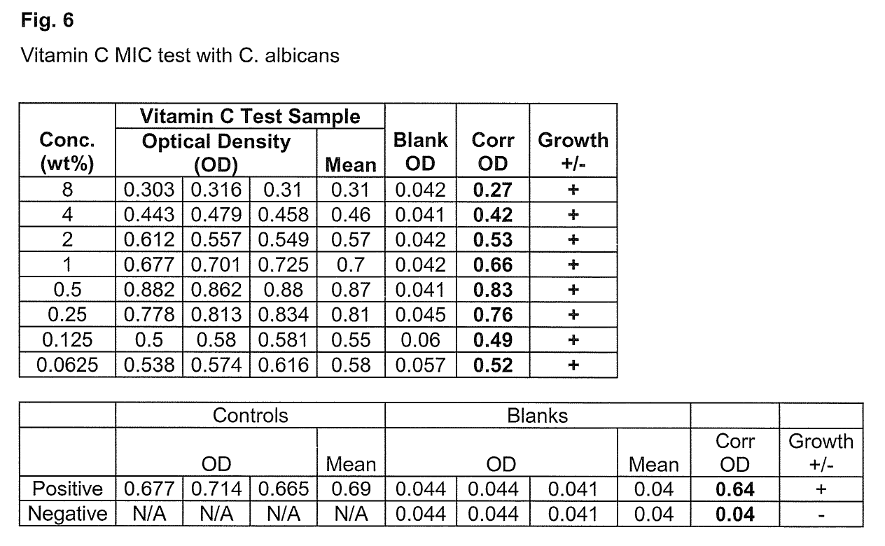

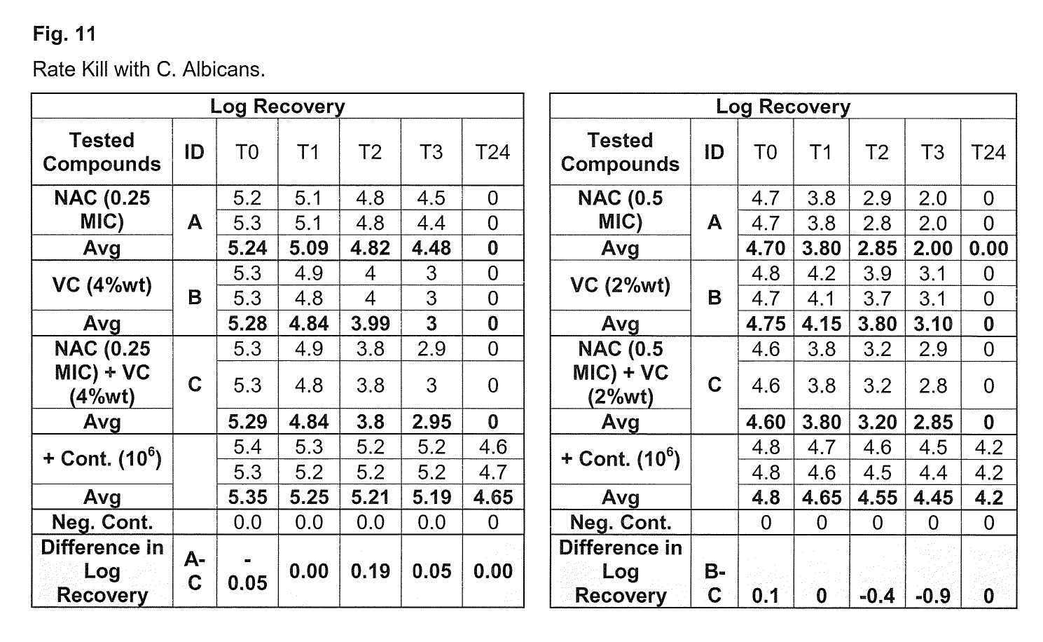

Note:
The MIC for Vitamin C against C. Albicans could not be determined; per experiments (see FIG. 6). The above solutions were then combined with C. Albicans and the log recovery of the C. Albicans was measured at times 0 hrs, 1, hr, 2, hrs, 3, hrs and 24 hrs. See FIG. 11 for results. The data does not show that NAC and Vitamin C solutions are synergistic against C. Albicans.

### pH Experiments

Further experiments were conducted to measure the effects of pH on NAC and Vitamin C formulations. In order to determine MIC and MBC (minimum bactericidal concentration) a National Committee on Clinical Laboratory Standards (NCCLS) micro-dilution procedure was followed. According to the procedure each formulation must be exposed to 6 log concentration of organism or the highest achievable concentration. In the current protocol 100 μL of MHB was mixed with 90 μL of formulation and 10 μL of log 8 organism or the highest achievable concentration. The concentration of the formulation was adjusted to obtain the required concentration in the final solution. The mixture was incubated at 37 degree C. for 16-24 hrs. After 16-24 hours the absorbance value was read at 600 nm. The obtained data was corrected by subtracting the appropriate blanks. Finally, the wells having an absorbance≧0.1 were marked + and <0.1 were marked −. The + symbol indicated growth while − symbol indicates no growth. The positive growth controls must have a corrective absorbance value of >0.5 and negative controls must have a corrected absorbance value of <0.1. In cases where the positive growth controls corrected absorbance is lower than 0.5, an alternate rule is utilized which is “absorbance<than 20% of positive growth control is marked as − growth, while absorbance≧than 20% of positive growth control is marked as + growth”. pH was adjusted to the stated value using NaOH or HCl.

Staphylococcus aureus (organism # 25923) was obtained from ATCC. L-Ascorbic Acid (Vitamin C) was used (Fisher Scientific, Catalogue # A61-25, Lot # 066251). N-acetyl cysteine (NAC) was used (Acros, Catalogue # 160280250, Lot # A0229576). A 4 wt % NAC solution in water was prepared at a pH of 4. A 4 wt % NAC solution in water was prepared at a pH of 6. A 4 wt % Vitamin C solution in water was prepared at a pH of 4. A 4 wt % Vitamin C solution in water was prepared at a pH of 6. These solutions were then serially diluted as necessary to obtain the required concentrations. A minimum concentration of NAC and Vitamin C that inhibited the growth of Staphylococcus aureus was found at each pH. As per experiments conducted:

NAC (pH=4) has a MIC of 2.0% (w/v) for S. aureus;

NAC (pH=4) has a MBC of 2.0% (w/v) for S. aureus;

NAC (pH=6) has a MIC that could not be determined with a 2.0% (w/v) starting point for S. aureus;

Vitamin C (pH=4) has a MIC of 0.5% (w/v) for S. aureus;

Vitamin C (pH=4) has a MBC of 1.0% (w/v) for S. aureus; and

Vitamin C (pH=6) has a MIC that could not be determined with a 2.0% (w/v) starting point for S. aureus;

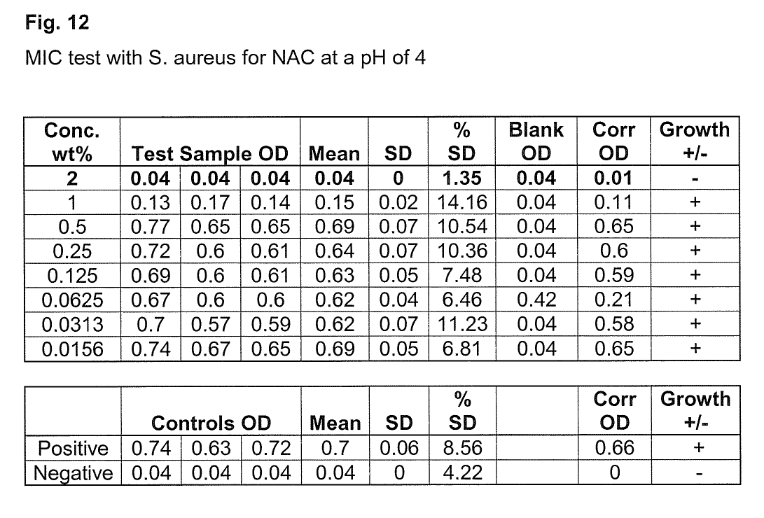

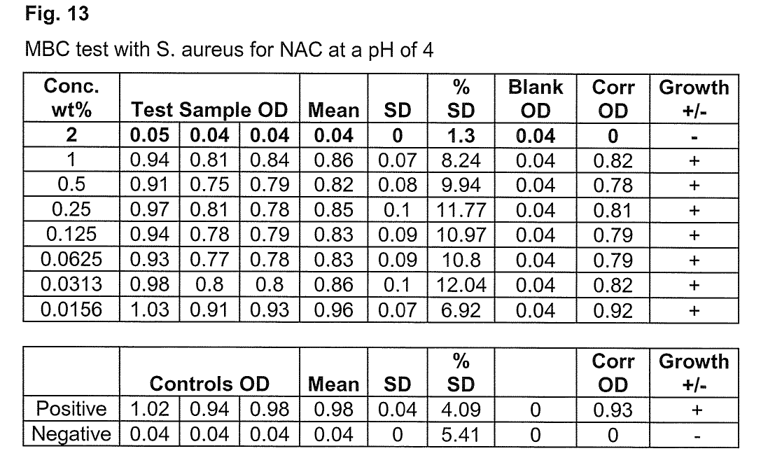

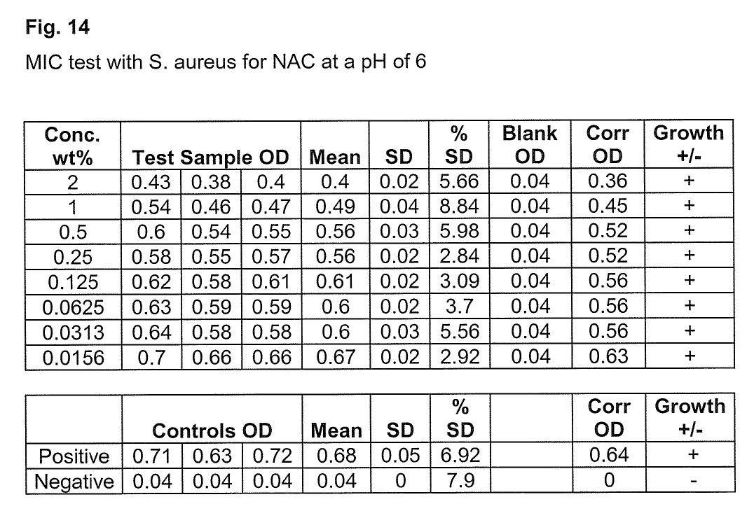

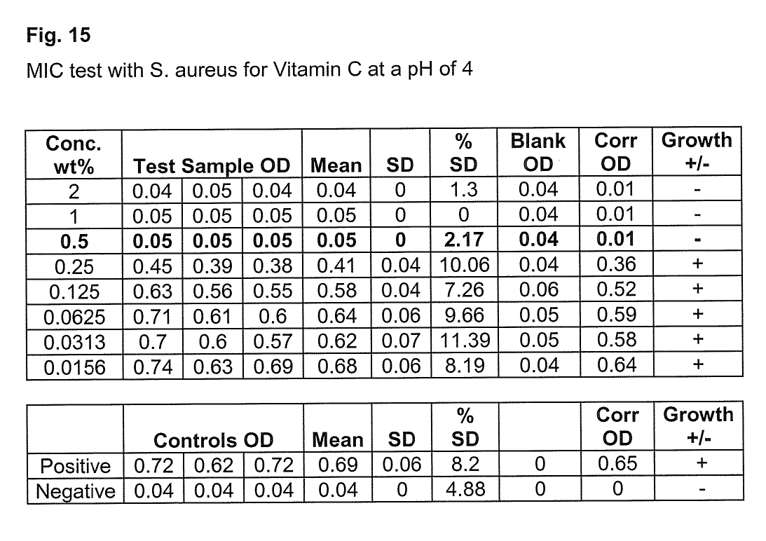

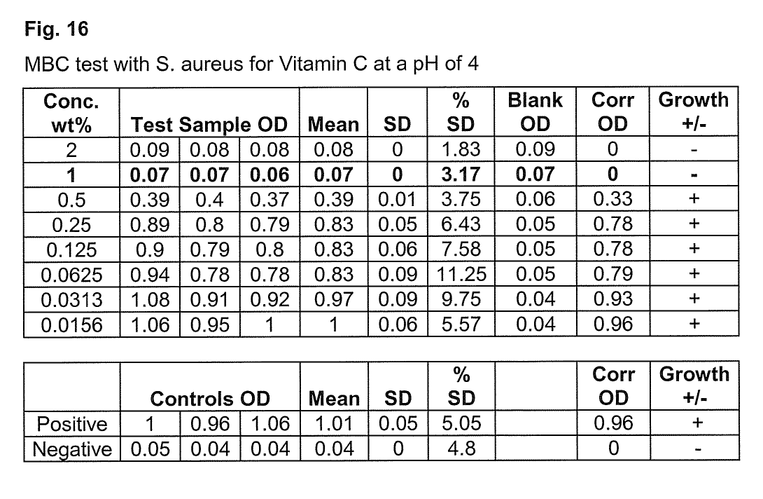

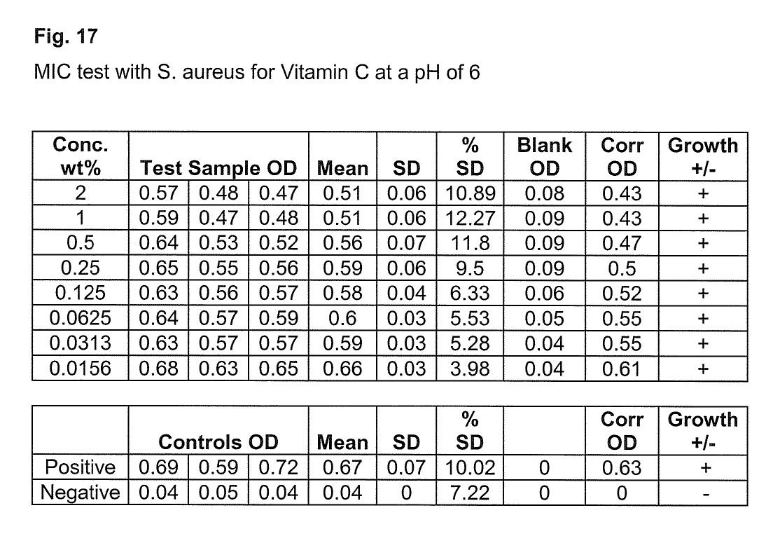

See FIGS. 12-17 for results.

Based on the above, a further experiment conducted was a screening experiment using checkerboard titration to assess if the combinations at a pH of 4 fall within a range having an FIC index value of ≦1. The method used was a NCCLS micro-dilution procedure. The results of this experiment are shown in FIG. 18. Based on the results the FIC index for NAC and Vitamin C at a pH of 4 is 0.6. The FIC index of 0.6 shows at least a partial synergy between NAC and Vitamin C at a pH of 4.

Accordingly, the effect of pH on the efficacy of the formulations against S. Aureus can be summarized as follows:

| | pH < 4 | pH = 4 | pH = 6 |
|---|---|---|---|
| | MIC Values | | |
| NAC | 0.25 wt % | 2 wt % | >2 wt % |
| VC | 0.25 wt % | 0.5 wt % | >2 wt % |
| | Synergy Assessment-FIC Values | | |
| NAC + VC | 0.8 | 0.6 | N/A |

From the above chart it is evident that with increase in pH the efficacy of the both NAC and Vitamin C reduces. In addition, it can be noted that the FIC value is not provided at pH 6, since a definite MIC could not be determined for both NAC & Vitamin C when 2 wt % (w/v) was utilized as the starting concentration.

### Anticoagulant Experiments

Experiments were conducted to assess the anticoagulant capacities of NAC, Vitamin C and combinations of NAC and Vitamin C via a Prothrombin Time (PT) Assay. A PT assay (TM-4339-063) was conducted using a Coagulation Analyzer to obtain PT instead of manually recording the PT.

L-Ascorbic Acid (Vitamin C) was used (Fisher Scientific, Catalogue # A61-25, Lot # 066251). N-acetyl cysteine (NAC) was used (Acros Organics, Catalogue # 160280250, Lot # A0229576). TriniCHECK 1 (Normal Control) was used (Trinity Biotech). TriniCHECK 2 (Abnormal Control) was used (Trinity Biotech). A KC4 Amelung Coagulizer was used (Trinity Biotech).

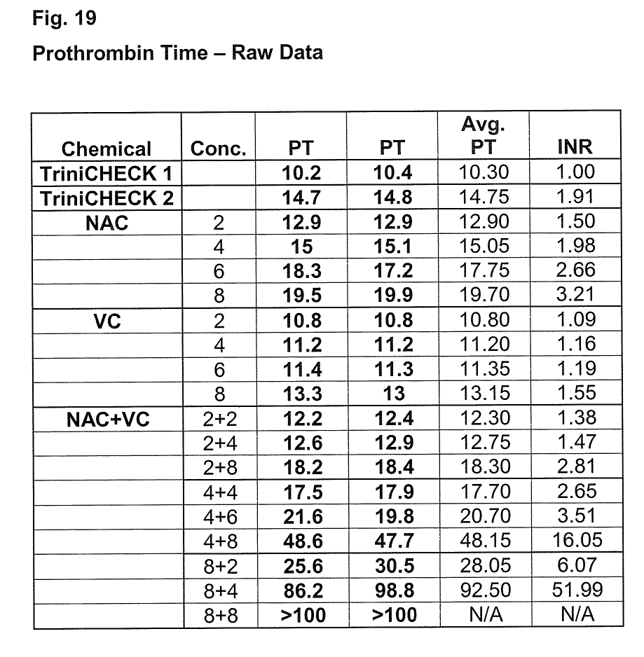

FIG. 19 shows the results (raw data) of the PT assay. The concentrations stated in the concentration column are the final concentrations of the reagents. TriniCHECK 1 is a normal control that provides the PT time in the range of what a normal blood sample would take to coagulate. TriniCHECK 2 is an abnormal control that provides the PT time above the range of what a normal blood sample would take to coagulate. INR (International Normalized Ratio) is a system established by the World Health Organization (WHO) and the International Committee on Thrombosis and Hemostasis for reporting the results of blood coagulation (clotting) tests. INR is calculated as:

INR=(PT_{test sample}/PT_{normal control})^{ISI}

ISI (International Sensitivity Index) indicates the sensitivity of individual thromboplastin. The value of ISI utilized herein was 1.89.

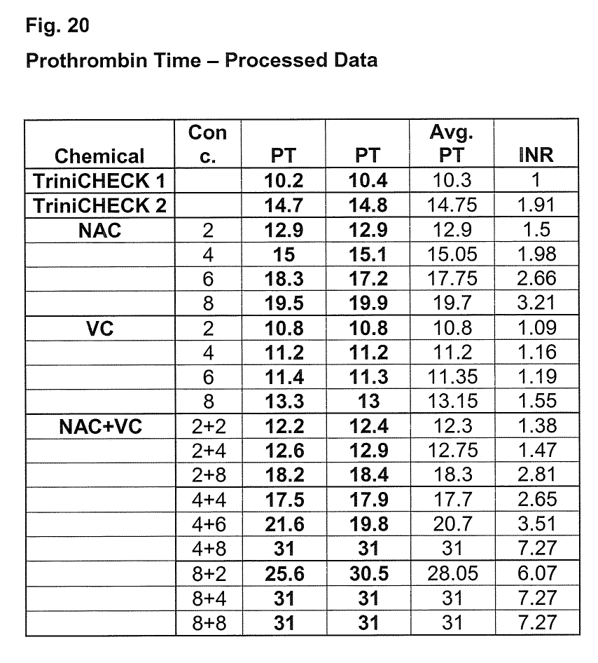

FIG. 20 shows the results (processed data) of the PT assay. All the PTs greater than 3× the TriniCHECK 1 (normal control) were replaced with 31 seconds. This was done for the following reasons: Instrument used does not provide reproducible readings at PTs greater than 45 seconds; PTs greater than 3× the normal control results in INR greater than 6 is the ISI is 1.89. Any INR value higher than 5.5 indicates very high anticoagulant capacity and any higher value is of very little or no clinical significance; and for better assessment of data.

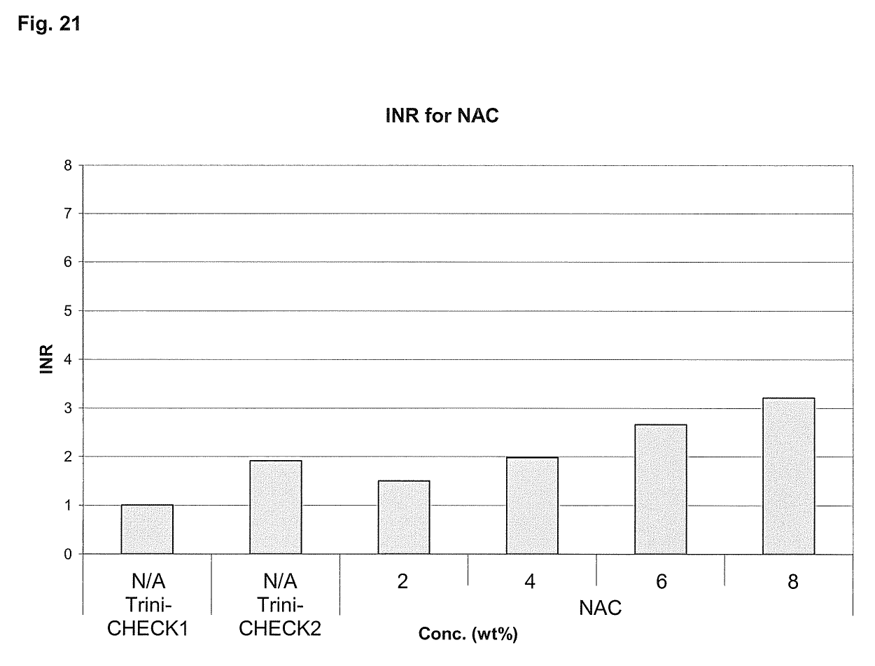

FIG. 21 shows the graph of the International Normalized Ratio (INR) for NAC from a Prothrombin Time (PT) Assay. From FIG. 21 it is evident that (within the tested range) that an increase in concentration of NAC results in an increase in INR.

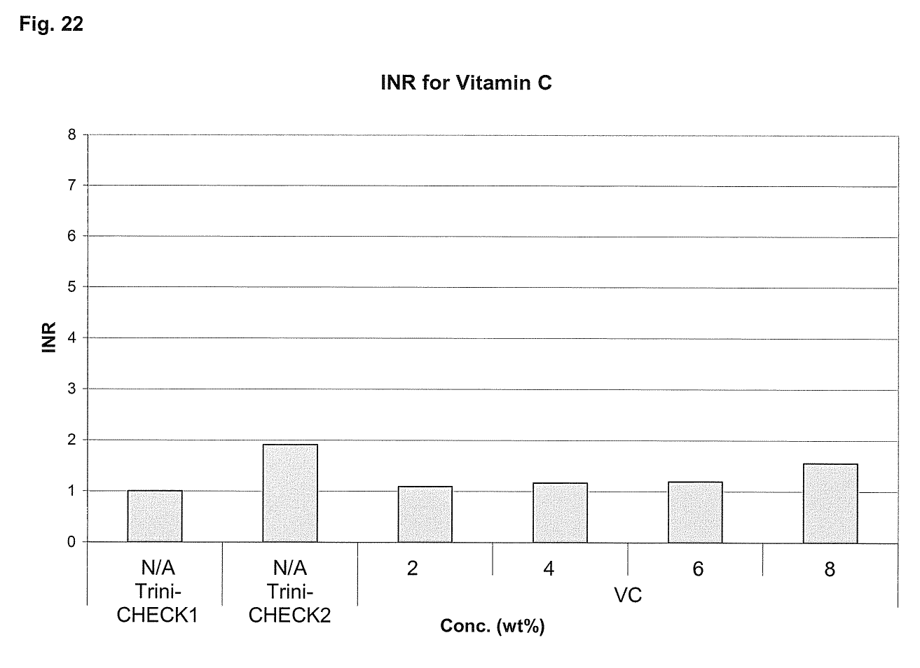

FIG. 22 shows the graph of the International Normalized Ratio (INR) for Vitamin C from a Prothrombin Time (PT) Assay. From FIG. 22 is it evident that (within the tested range) an increase in concentration of Vitamin C results in no significant increase in INR.

FIG. 23 shows the graph of the International Normalized Ratio (INR) for combined NAC and Vitamin C formulations from a Prothrombin Time (PT) Assay. From FIG. 23, and comparing results from FIGS. 21 and 22, it is evident that (within the tested range) that the addition of Vitamin C significantly, and surprisingly, enhances the anticoagulant activity of NAC. Significant enhancement in INR is observed when, for example, i) 8 wt % NAC is mixed with 2, 4 and 8 wt % Vitamin C; and ii) 4 wt % NAC is mixed with 6 and 8 wt % Vitamin C.

From the foregoing, it should be clear that the present disclosure may be embodied in forms other than those discussed above; the scope of the present disclosure should be determined by the following claims and not the detailed discussion presented above.

1. A disinfectant composition comprising N-Acetyl Cysteine (NAC) and Vitamin C in solution,
wherein the NAC is at a concentration of at least 0.01% (w/v) and less than 12% (w/v), the Vitamin C is at a concentration of at least 0.01% (w/v) and less than 12% (w/v), and the disinfectant composition has a pH of 6 or less.

2. The composition of claim 1, wherein the solution comprises between 0 and 10% (v/v) ethanol and water.

3. The composition of claim 1, wherein the solution comprises saline.

4. The composition of claim 1, wherein the NAC is at a concentration of at least 0.1% (w/v) and less than 2.5% (w/v), wherein the Vitamin C is at a concentration of at least 0.1% (w/v) and less than 2.5% (w/v).

5. The composition of claim 1, wherein the disinfectant composition has a pH of 4 or less.

6. A disinfectant composition comprising N-Acetyl Cysteine (NAC) and Vitamin C in a dry or partially hydrated formulation that, upon reconstitution with a solution, forms a disinfectant composition wherein the NAC is at a concentration of at least 0.01% (w/v) and less than 12% (w/v), the Vitamin C is at a concentration of at least 0.01% (w/v) and less than 12% (w/v), and the disinfectant composition has a pH of 6 or less.

7. A disinfectant composition comprising N-Acetyl Cysteine (NAC) and Vitamin C in solution,
wherein the NAC is at a concentration of at least 0.01% (w/v) and less than 12% (w/v), the Vitamin C is at a concentration of at least 0.01% (w/v) and less than 12% (w/v), the disinfectant composition has a pH of 6 or less, the disinfectant composition is packaged in a sterile, non-pyrogenic form, the solution is water, and the disinfectant composition has an osmolarity of from 240-500 mOsM/Kg.

8. A lock flush composition comprising N-Acetyl Cysteine (NAC) and Vitamin C in solution,
wherein the NAC is at a concentration of at least 0.01% (w/v) and less than 12% (w/v), the Vitamin C is at a concentration of at least 0.01% (w/v) and less than 12% (w/v), the lock flush composition has a pH of 6 or less, the lock flush composition is packaged in a sterile, non-pyrogenic form, and the lock flush composition is biocompatible for use in in-dwelling access catheters, urinary catheters, nasal tubes and throat tubes.

9. The method for disinfecting a surface by contacting the surface with a disinfectant solution comprising N-Acetyl Cysteine (NAC) and Vitamin C in solution,
wherein the NAC is at a concentration of at least 0.01% (w/v) and less than 12% (w/v), the Vitamin C is at a concentration of at least 0.01% (w/v) and less than 12% (w/v), the disinfectant composition has a pH of 6 or less.

10. The method of claim 9, wherein the surface is a conduit selected from the group consisting of a conduit carrying sterile fluids, blood, or plasma, a catheter, an airway support device, a port, and a subcutaneous port.

11. The method of claim 9, wherein the solvent is water.

12. The method of claim 9, wherein the NAC is at a concentration of at least 0.1% (w/v) and less than 1% (w/v), wherein the Vitamin C is at a concentration of at least 0.1% (w/v) and less than 1% (w/v), and wherein the disinfectant composition has a pH of 4 or less.

13. The method of claim 10, wherein contacting the conduit with the disinfectant solution is accomplished by locking, flushing or coating the conduit with the disinfectant solution.

14. A method for disinfecting a catheter comprising:
introducing a disinfectant solution into an interior lumen of the catheter, wherein the disinfectant solution comprises N-Acetyl Cysteine (NAC) and Vitamin C in solution, wherein the NAC is at a concentration of at least 0.01% (w/v) and less than 12% (w/v), wherein the Vitamin C is at a concentration of at least 0.01% (w/v) and less than 12% (w/v) wherein the disinfectant composition has a pH of 6 or less;
holding the disinfectant solution within the lumen for a selected period of time; and
removing the disinfectant solution from the interior lumen.

15. An anticoagulant composition comprising N-Acetyl Cysteine (NAC) and Vitamin C in solution,
wherein the NAC is at a concentration of at least 0.01% (w/v) and less than 12% (w/v), the Vitamin C is at a concentration of at least 0.01% (w/v) and less than 12% (w/v), and the disinfectant composition has a pH of 6 or less.

16. The composition of claim 15, wherein the NAC is at a concentration of at least 2% (w/v) and less than 6% (w/v), wherein the Vitamin C is at a concentration of at least 6% (w/v) and less than 10% (w/v).
